(19) United States
(12) Patent Application Publication
(10) Pub. No.: US 2005/0121057 A1
(43) Pub. Date: Jun. 9, 2005

(54) System for Disinfecting Shopping Carts

(75) Inventors: Christopher M. Knowlton, Pinehurst, NC (US); Robert J. O'Hara, Pinehurst, NC (US); Michael J. Roden, Prescott, AZ (US); James R. Roden, Scottsdale, AZ (US)

Correspondence Address: CHRISTOPHER J. KULISH, ESQ, HOLLAND & HART LLP, P. O. BOX 8749, DENVER, CO, 80201-8749, US

(73) Assignee: REDFIELD ENGINEERING COMPANY, LLC, Prescott, AZ (US)

(21) Appl. No.: 10/707,379

(22) Filed: Dec. 9, 2003

(51) Int. Cl. B08B 3/02

(52) U.S. Cl. 134/68; 134/61; 134/66; 134/72; 134/123

(57) ABSTRACT

The present invention provides a system for disinfecting shopping carts. In one embodiment, the system comprises a treatment station for applying a disinfectant to a shopping cart and a conveyor system for transporting carts relative to the treatment station at two different speeds so as to effect a nesting operation. In another embodiment, the system comprises a treatment station that is comprised of a first modular unit and a second modular unit that each perform different functions but can readily joined to one another to form a treatment station with the desired features for a particular application. In yet another embodiment, certain features of the modular units are symmetrical relative to a lateral plane to facilitate the joining of the units to one another in different orders. A further embodiment comprises a treatment station with a molded structure. In yet another embodiment, the system comprises a treatment station for applying a liquid disinfectant to a shopping cart and that is designed to be resistant to freezing in cold environments.

### FIELD OF THE INVENTION

The present invention is directed to shopping carts and, in particular, to the disinfection of shopping carts.

### BACKGROUND OF THE INVENTION

Presently, shopping carts are rarely disinfected. Consequently, a shopping cart can present a health risk to a shopper using the shopping cart. To elaborate, organisms that present health risks can be transferred to a shopping cart from leaking meat and poultry packages, poorly fitting baby diapers, a shopper's hand or nose, and the like. These organisms can then be transmitted from the shopping cart to a shopper that subsequently uses the cart.

To address the health risk associated with the transfer of an organism from a shopping cart to a shopper, various systems for cleaning or disinfecting shopping carts have been devised. Many of these systems include a cleaning or disinfecting station and a transport device for moving shopping carts through the station. One system includes a mobile trailer that supports a cleaning or disinfecting station and a transport device. Another system comprises a treatment station that has multiple treatment zones and a transport system for moving the carts through the treatment zones.

### SUMMARY OF THE INVENTION

The present invention provides a system for disinfecting shopping carts that may be contaminated with an organism that presents a health risk to shoppers that utilize the carts.

In one embodiment, the system comprises a treatment station for applying a disinfectant to a shopping cart and a conveyor system for transporting shopping carts relative to the treatment station at two different speeds to facilitate a nesting operation. To elaborate, in many instances it is desirable to provide a string of nested shopping carts to the system for processing. However, it is also desirable that the shopping carts be “un-nested” or separated from one another so that those surfaces of the carts in a string of nested shopping carts that would not be sufficiently exposed for receiving the disinfecting treatment are exposed in time to receive the disinfecting treatment. In one embodiment, the conveyor system comprises two conveyors that operate at different speeds to facilitate the “unnesting” of a shopping cart from a string of nested shopping carts. To elaborate, the conveyor system comprises a first conveyor for moving a string of nested shopping carts at a first speed and a second conveyor for moving a shopping cart at a second speed that is greater than the first speed. Initially, a string of shopping carts is placed on the first conveyor. When the lead shopping cart of the string of nested shopping carts is transferred from the first conveyor to the second conveyor, the difference in speeds of the two conveyors causes a force to be applied the lead cart that separates the lead shopping cart from the following shopping cart.

In other instances, it is desirable to create a string of nested shopping carts from carts that have been treated. Consequently, in another embodiment of the system, the conveyor system comprises two conveyors that operate at different speeds to facilitate the “nesting” of one treated shopping cart with another treated shopping cart. More specifically, the conveyor system comprises a first conveyor for moving an “un-nested” and treated shopping cart at a first speed and a second conveyor for moving another treated shopping cart at a second speed that is less than the first speed. In operation, the first and second conveyors are used to form a string of nested shopping carts. To elaborate, assume that a first shopping cart is on the first conveyor and a second shopping cart is on the second conveyor. When the first cart is transferred from the first conveyor to the second conveyor, the difference in speeds of the two conveyors forces the first cart towards the second cart such that the first cart nests with the second cart.

In yet other instances, it is desirable to: (a) “un-nest” a string of nested shopping carts so that those surfaces of the carts in the string of nested shopping carts that would not be sufficiently exposed for receiving the disinfecting treatment are exposed in time to receive the disinfecting treatment; and (b) create a string of nested shopping carts from carts that have been treated. In one embodiment, two conveyors are utilized, one conveyor being a high speed conveyor and the other conveyor being a low speed conveyor that extends beyond the ends of the low speed conveyor. The conveyors are situated so as that a shopping cart is: (a) initially engaged by the low speed conveyor; (b) then transitioned from the low-speed conveyor to the high speed conveyor to facilitate separation of the shopping cart from a string of nested shopping carts and thereby expose surfaces of the shopping cart in time to receive the disinfecting treatment; and (c) then transitioned from the high speed conveyor back to the low speed conveyor to nest the shopping cart with any previously treated shopping cart that is being transported by the low speed conveyor. In yet a further embodiment, three conveyors are utilized, two low speed conveyors that are separated from one another and a high-speed conveyor that extends between the two low speed conveyors. In this embodiment, one of the low speed conveyors and the high speed conveyor are used to facilitate the “unnesting” of a lead shopping cart in a string of shopping carts. The high speed conveyor is also used in conjunction with the other low speed conveyor to facilitate the “nesting” of one treated shopping cart with another treated shopping cart. In another embodiment of the system, the conveyor system comprises four conveyors, two conveyors for un-nesting a string of nested shopping carts and the other two conveyors for forming a string of nested carts.

Another embodiment of a system for disinfecting shopping carts comprises a modular treatment station and a transport system for moving a shopping cart relative to the treatment station. The treatment station defines an entry for receiving a shopping cart, an exit for providing a treated cart, and a pathway extending between the entry and the exit and along which a shopping cart travels during operation of the system. The treatment station is comprised of first and second modular units that each define a portion of the pathway and that each have a ground engagement surface. The modularity of the first and second modular units arises from the relationship of the ground structure of each of the modular units to the pathway defining structure of each of the modular units. To elaborate, these relationships are such that when the ground structures of both of the modular units are in contact with a flat surface, the portions of the pathway defined by the two units can be readily aligned with one another, thereby facilitating the joining of one unit to another. The modularity feature allows modular units that perform different functions to be designed and then readily combined with one another to realize a treatment station with the desired features for a particular application.

In another embodiment, modular units that have a symmetrical interface structure are employed to realize a treatment station. The symmetrical interface structure allows the modules to be concatenated with one another in a number of different sequences. For example, if a first modular unit is adapted to apply a liquid disinfectant to a shopping cart and a second modular unit is adapted to dry a shopping cart after a liquid disinfectant has been applied to the shopping cart, the symmetrical interface structure of each of the modules allows a functional treatment station to be realized in which the left-to-right order of the modules from a given view point is first module second module or second module first module. Stated differently, the symmetry characteristic of each of the modules allows: (a) a treatment station to be realized that, when the station is viewed from the side, receives shopping carts from the right hand side of the treatment station; or (b) a treatment station to be realized that, when the station is viewed from the same side, receives shopping carts from the left hand side of the treatment station.

A further embodiment of the system comprises a treatment station for applying a disinfectant to a shopping cart that comprises a molded structure and a transport system for moving a shopping cart relative to the treatment station. In one embodiment, the molded structure forms two or more elements of the treatment station and thereby reduces the number of components that would otherwise be required to realize the station. For example, in one embodiment, the molded structure comprises a single-piece enclosure that defines at least a portion of the pathway along which a shopping cart travels during operation of the system and a reservoir for holding the liquid disinfectant that is applied to the shopping carts. In another embodiment, the molded section forms an element that is less susceptible to damage or vandalism. For example, in one embodiment, the molded structure forms an air nozzle that is less susceptible to damage or vandalism than, for instance, metal nozzles that are attached to a supporting structure via a threaded coupling mechanism.

Yet another embodiment of the system comprises a treatment station that employs a low-pressure pump that is used to move disinfectant from a reservoir to an application structure that dispenses the disinfectant onto a shopping cart during operation of the system and a transport system for moving shopping carts relative to the treatment station. In one embodiment, the low-pressure pump comprises a centrifugal pump that is located, during operation of the system, at substantially the same elevation as the reservoir. This orientation of the centrifugal pump relative to the reservoir facilitates a “gravity drain” design for the system in which the disinfectant in the circuit formed by the reservoir, pump, and application structure, during operation of the system, is drawn back to the reservoir by gravity after the pump is deactivated. This “gravity drain” facilitates use of the system in cold weather applications. To elaborate, the gravity drain characteristic allows disinfectant to drain back to the reservoir when the centrifugal pump is inoperative. A heater located in the reservoir then keeps the disinfectant from freezing. In contrast, if there was not a gravity drain and a significant amount of disinfectant remained in the circuit after the pump was deactivated, the entire circuit would need to be heated to prevent the disinfectant from freezing in the circuit and rendering the system inoperative.

### BRIEF DESCRIPTION OF THE DRAWINGS

FIGS. 1A-1D respectively illustrate front, back, entry, and exit side views of an embodiment of a shopping cart treatment system that is configured for a right-hand cart-entry;

FIG. 2 is a cross-sectional view of the floor path and portions of the conveyor system associated with the transport system of the embodiment of the system illustrated in FIGS. 1A-1D;

FIGS. 3A-3C respectively illustrate top, side and enlarged side, free body diagrams of elements of the conveyor system associated with the shopping cart treatment system shown in FIGS. 1A-1D;

FIGS. 4A-4B illustrate the operation of the staging bar assembly associated with the conveyor system shown in FIGS. 2A-2B;

FIGS. 5A-5C respectively illustrate a pair of modular units separated from one another and a baffle, the pair of modular units joined to one another with a baffle interposed between the units, and an alternative embodiment of a baffle;

FIGS. 6A-6B illustrate the use of symmetrical and modular units to realize treatment systems that are respectively capable of receiving untreated shopping carts from the right-hand side and the left-hand side;

FIGS. 7A-7B respectively are lateral and longitudinal cross-sectional views of an embodiment of a disinfectant solution delivery system suitable for use in a shopping cart treatment station;

FIGS. 8A-8B respectively are lateral and longitudinal cross-sectional views of another embodiment of a disinfectant solution delivery system suitable for use in a shopping cart treatment station;

FIG. 9 is a longitudinal cross-sectional view of a treatment station comprises of a recycling liquid disinfectant delivery system, a non-recycling liquid disinfectant delivery stations, and a drying system;

FIGS. 10A-10B respectively are lateral and longitudinal cross-sectional views of a dryer delivery system suitable for use in a shopping cart treatment station;

FIG. 11 is a longitudinal cross-sectional view of a treatment station comprised of a liquid disinfectant delivery system and a dryer system;

FIG. 12 illustrates an embodiment of a wheeled container for use in removing spent liquid disinfectant from a liquid disinfectant delivery system and providing unused liquid disinfectant to the system;

FIG. 13 illustrates an embodiment of a brush system for removing particles from the wheels of shopping carts that is suitable for use with a shopping cart disinfecting system.

### DETAILED DESCRIPTION

The present invention is directed to a system for disinfecting shopping carts. Generally, the system is comprised of a treatment station for applying a disinfecting treatment to a shopping cart and a transport system for moving a shopping cart relative to the treatment station.

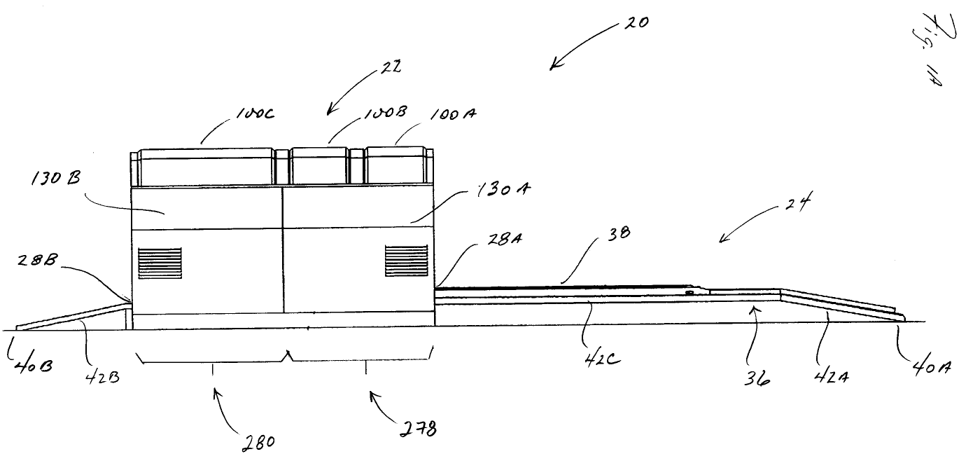

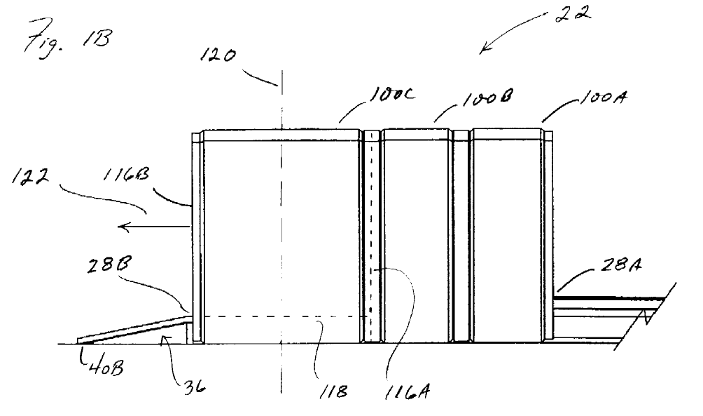

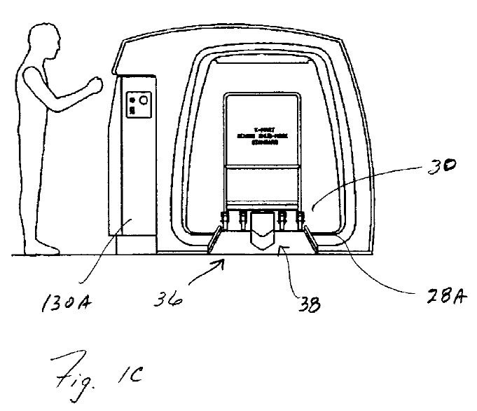

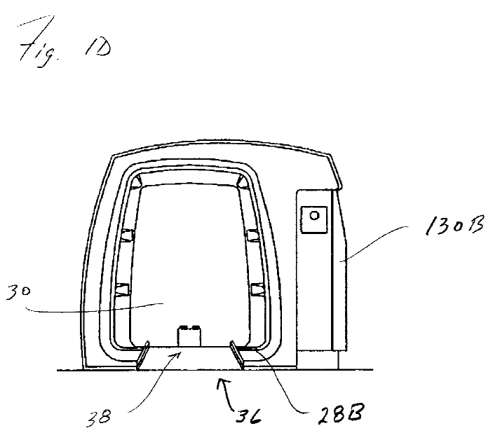

FIGS. 1A-1D illustrate an embodiment of a system for disinfecting shopping carts, hereinafter referred to as system 20. The system 20 comprises a treatment station 22 for applying a disinfecting treatment to a shopping cart and a transport system 24 for moving a shopping cart relative to the treatment station.

The treatment station 22 defines an entry threshold 28A, exit threshold 30B, and a pathway 30 that extends between the entry threshold 28A and exit threshold 28B and through which the shopping carts pass during treatment.

The transport system 24 is capable of: (a) receiving a single shopping cart and moving the shopping cart over the pathway 30 defined by the treatment station 22; (b) receiving multiple shopping carts that are sufficiently separated from one another so that no further separation is needed to sufficiently expose the surfaces of the cart to the disinfecting treatment provided by the treatment station 22 and moving each of the shopping carts over the pathway 30 defined by the treatment station 22; (c) receiving a string of nested shopping carts, separating the nested shopping carts from one another so that the surfaces of each of the shopping carts that would not otherwise be sufficiently exposed for receiving a disinfecting treatment are sufficiently exposed in time to receive the disinfecting treatment, nesting treated shopping carts with one another, and moving each of the shopping carts over the pathway 30 defined by the treatment station 22. It should be appreciated that when the term “nested” or a similar term is used with respect to shopping carts that have not been treated, the term means that the shopping carts are not sufficiently separated from one another so that the surfaces of each of the shopping carts is sufficiently exposed for receiving a disinfecting treatment. Consequently, nested shopping carts may or may not be in physical contact with one another. Concomitantly, the term “un-nesting” means that the untreated shopping carts are separated from one another such that the surfaces of each shopping cart are sufficiently exposed for receiving a disinfecting treatment. It should also be appreciated that when the term “nesting” or similar term is used with respect to treated shopping carts, the term means that the carts are sufficiently close to one another that the surfaces of each of the shopping carts are not sufficiently exposed for receiving a disinfecting treatment.

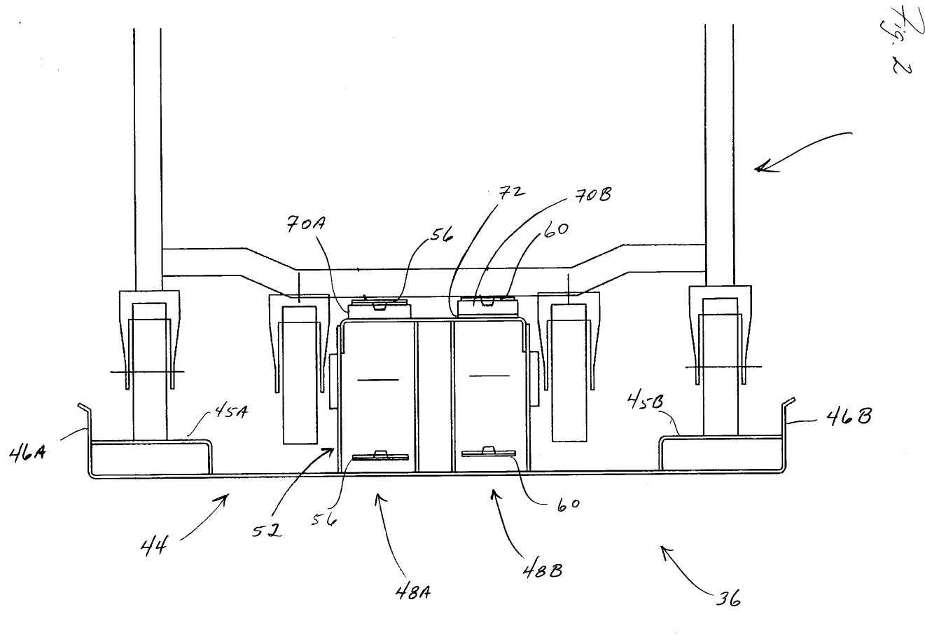

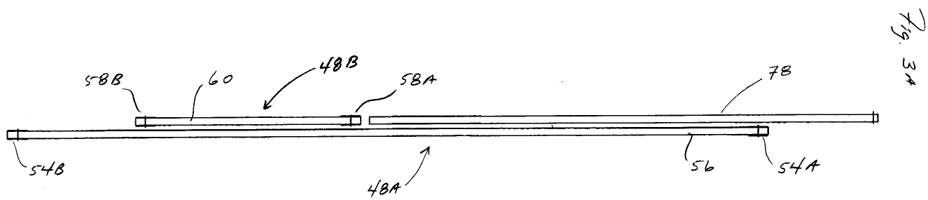

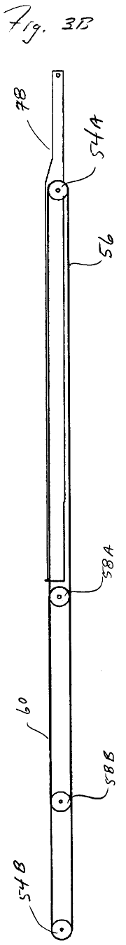

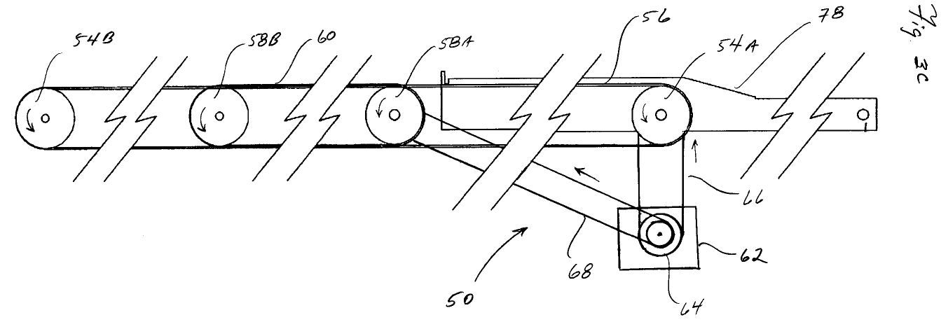

The transport system 24 comprises a floor structure 36 that defines the path over which a shopping cart travels during processing by the system 20 and a conveyor system 38 for moving a shopping cart over at least a portion of the floor structure 36. The floor structure 36 extends from a first floor end 40A to a second floor end 40B. Further, the floor structure 36 comprises an incline section 42A up which a shopping cart is moved prior to treatment, a decline section 42B down which a shopping cart rolls after treatment, and a raised section 42C that extends between the incline section 42A and decline section 42B. With reference to FIG. 2, the floor structure 36 comprises a bottom surface 44 that engages wheels associated with a shopping cart and a pair of side rails 46A, 46B that serve to guide a shopping cart over the floor structure 36. Associated with the bottom surface 44 are a pair of raised platforms 45A, 45B that support the rear wheels of a shopping cart when the shopping cart has been engaged by the conveyor system 36 to assure that the axes of the front and rear wheels remain in a substantially level plane that facilitates nesting operations.

With continuing reference to FIG. 2, the conveyor system 38, in addition to moving a shopping cart over at least a portion of the floor structure, facilitates: (a) the separation of nested shopping carts from one another so that the surfaces of each shopping cart are sufficiently exposed in time to receive the disinfecting treatment; and (b) the nesting of treated shopping carts with one another. The separation of nested shopping carts and nesting of treated shopping carts is facilitated by using two conveyor belts that, during operation, move at different speeds. To elaborate, the separation of nested shopping carts is facilitated by causing the lead shopping cart of a group of nested shopping carts to transition from the slower of the two conveyor belts to the faster of the first two conveyor belts. The difference in the speed facilitates the separation of the lead shopping cart from the following shopping cart. The nesting of treated shopping carts is facilitated by causing a shopping cart to transition from the faster of the two conveyor belts to the slower of the two conveyor belts. If another shopping cart is already on the slower of the first and second conveyor belts, the difference in speed forces the shopping cart that is transitioning from the faster conveyor belt to the slower conveyor belt to nest with the shopping cart that is already on the slower conveyor belt.

With continuing reference to FIG. 2 and reference to FIGS. 3A-3C, the conveyor system 38 comprises: (a) first and second conveyor belt systems 48A, 48B; (b) a drive system 50 for transmitting power to the first and second conveyor belt system 48A, 48B so that a belt associated with the first convey belt system 48A moves at a first speed and a belt associated with the second conveyor belt system 48B moves at a second speed that is greater than the first speed; and (c) a housing 52 for supporting elements of the first and second conveyor belt systems 48A, 48B.

The first conveyor belt system 48A comprises a pair of grooved pulleys 54A, 54B and a belt 56 that extends between the pulleys 54A, 54B and is used to engage a shopping cart. Similarly, the second conveyor 48B comprises a pair of grooved pulleys 58A, 58B and a belt 60 that extends between the pulleys 58A, 58B and is used to engage a shopping cart. Tensioning of each of the belts 56, 60 is accomplished with an idler pulley (not shown). As shown in FIG. 3A, the first conveyor belt system 48A extends from a first end that is defined by the grooved pulley 54A to a second end that is defined by the grooved pulley 54B. Similarly, the second conveyor belt system 48B extends from a first end that is defined by the grooved pulley 58A to a second end that is defined by the grooved pulley 58B. Further, the first and second ends of the second conveyor belt system 48B are located between the first and second ends of the first conveyor belt system 48A.

The drive system 50 comprises: (a) an electric motor 62 that is used to provide the power that is used to drive the first and second conveyor belt systems 48A, 48B; (b) a two-groove pulley 64 that is operatively attached to the drive shaft of the electric motor 62; (c) a first drive belt 66 for transmitting power from the electric motor 62 to the pulley 54A of the first conveyor belt system 48A to drive the belt 56 at the first speed; and (d) a second drive belt 68 for transmitting power from the electric motor 62 to the pulley 58A of the second conveyor belt system 48B to drive the belt 60 at the second speed. The electric motor 62, two-groove pulley 64, first drive belt 66 and second drive belt 66 operate so that the pulleys 54A, 58A each rotate in a counter-clockwise direction. As a consequence, the belt 56 and the belt 60 each rotate in a counter-clockwise direction. Further, any shopping cart engaged by the belt 56 will be driven away from the first floor end 40A and towards the second floor end 40B. Likewise, any shopping cart engaged by the belt 60 will be driven away from the first floor end 40A and towards the second floor end 40B. Other drive systems for moving two conveyor belts at different speeds are feasible. For example, in another embodiment, two electric motors are utilized, one to drive the first conveyor belt system 48A and the other to drive the second conveyor belt system 48B. In another embodiment, the drive shaft of an electric motor is coupled to a pulley of a conveyor belt system without the use of a belt or chain.

The housing 52 provides mounting surfaces for the pulleys associated with the first and second conveyor belt systems 48A, 48B. In addition, the housing 52 also supports first and second low-friction surfaces 70A, 70B on which the belts 56, 60 respectively ride. The pulleys associated with the first and second conveyor systems 48A, 48B and the first and second low-friction surfaces 70A, 70B are mounted to the housing 52 such that when the system 20 is operational, the belt 60 is a greater distance from the bottom surface 44 than the belt 56. In the illustrated embodiment, the pulleys 54A, 54B, 58A and 58B are all substantially the same size. Further, the pulleys 54A, 54B, 58A, 58B are mounted to the housing 52 such that when the system 20 is operational, the axes of rotation of the pulleys 58A, 58B are a greater distance from the bottom surface 44 than the axes of rotation of the pulleys 54A, 54B. Moreover, the low-friction surfaces 70A, 70B are of substantially the same thickness. As a consequence, a shim 72 is located between the housing 52 and the second low-friction surface 70B to assure that the belt 60 is supported at the noted greater distance. Other approaches for positioning the belt 60 at a greater distance from the bottom surface 44 than the belt 56 are feasible. For instance, in one embodiment, pulleys of different diameters and low-friction surfaces of different thickness are employed.

Based on the foregoing, it should be appreciated that a shopping cart being moved by the conveyor system 38 will move in a direction away from the pulley 54A and towards the pulley 54B. Further, in moving in this direction, the shopping cart will be subjected to: (a) a transition from the belt 54 to the belt 60 at a point adjacent to the pulley 58A; and (b) a transition from the belt 60 to the belt 54 at a point adjacent to the pulley 58B. When a string of nested shopping carts is being processed by the system 20, the transition adjacent to the pulley 58A results in a force being applied to a lead shopping cart in the string of nested shopping carts that facilitates separation of the lead shopping cart from the following shopping cart. There is also a slight rotation of the lead shopping cart at the transition adjacent to the pulley 58A due to the difference in heights of the belts 54, 60 that also facilitates the separation of the lead shopping cart from the following shopping cart. Separation of nested shopping carts is desirable so that the surfaces of each of the nested shopping carts that may not otherwise be sufficiently exposed for receiving the disinfecting treatment are sufficiently exposed in time to receive the disinfecting treatment. Consequently, the transition point adjacent to the pulley 58A is located relative to the treatment station 22 so that nested shopping carts are separated from one another such that the surfaces of each of the nested shopping carts that may not otherwise be sufficiently exposed for receiving the disinfecting treatment are sufficiently exposed in time to receive the disinfecting treatment. The nesting of treated shopping carts is also desirable. Consequently, the transition point adjacent to the pulley 58B is located relative to the treatment station 22 so that a treated shopping cart is forced to nest with any previously treated shopping cart that is being transported by the conveyor system 38.

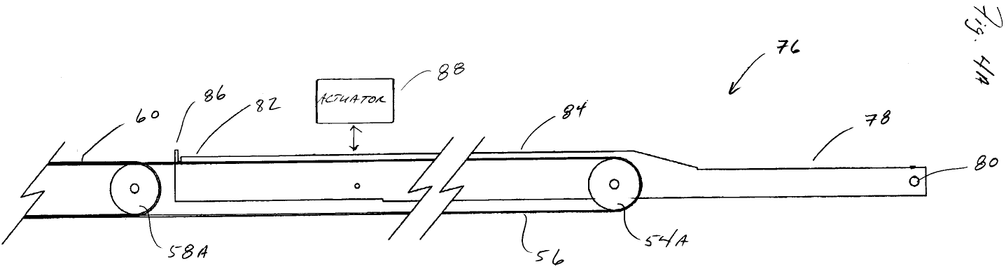

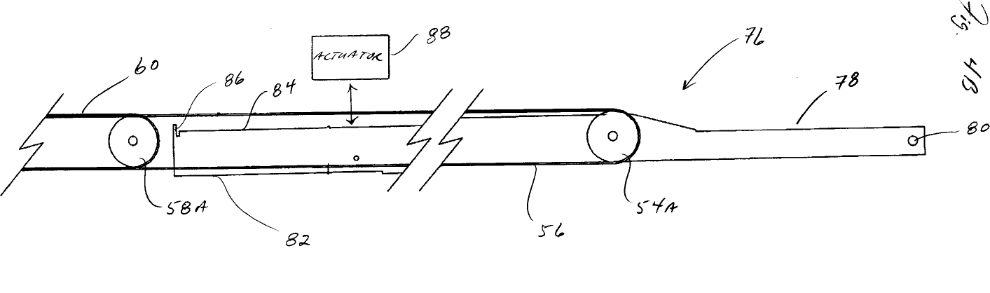

With reference to FIGS. 4A-4B, the conveyor system 38 further comprises a staging bar assembly 76 that allows an operator to: (a) position one or more shopping carts over the belt 56 of the first conveyor system 48A but prevent the one of more shopping carts from coming into contact with the belt 56; and (b) bring one or more shopping carts that have been positioned over the belt 56 but prevented from coming into contact with the belt 56 into contact with the belt 56. Typically, the staging bar assembly 76 is used to facilitate batch processing of shopping carts by initially allowing a number of shopping carts to be positioned over but separated from then belt 56 and then allowing all of these shopping carts to be brought into contact with the belt 56 at substantially the same time.

The staging bar assembly 76 comprises a staging bar 78 with a first end 80 that is pivotally attached to the floor structure 36, a second end 82, an upper surface 84 for engaging one or more shopping carts, and a hook 86 for engaging a surface associated with one shopping cart. The staging bar assembly 76 further comprises an actuator 86 that allows an operator to rotate the staging bar 78 between a position at which the staging bar 78 prevents a cart or carts from contacting the belt 56 and a position at which the staging bar 78 allows a cart or carts to contact the belt 56. The actuator 86 can be either a manual device or a device that employs electrical, hydraulic and/or pneumatic componentry. FIG. 4A illustrates the staging bar 78 in the position that prevents one or more shopping carts that are in contact with the upper surface 84 of the staging bar 78 from coming into contact with the belt 56. In this position, the upper surface 84 prevents the surface of a shopping cart that would otherwise come into contact with the belt 56 from contacting the belt 56. In this position, the hook 86 also prevents the shopping cart that is located closest to the hook 86 from coming into contact with the belt 56 by sliding off of the second end 82 of the staging bar 76. As a consequence, any other carts that are engaged by the staging bar 78 are also prevented from coming into contact with the belt 56 by sliding off of the second end 82 of the staging bar 76. In FIG. 4B, the staging bar 78 is in the position that allows shopping carts to engage the belt 56. Other staging bar assemblies are feasible. For instance, a staging bar assembly with a staging bar that is linearly translated, rather than rotated, is feasible. Also feasible is a staging bar assembly that does not prevent a shopping cart or carts from coming into contact with the belt 56 but prevents any carts from being moved by the belt 56.

The conveyor system 38 is capable of being used in several different ways. For example, the conveyor system 38 is capable of being used to: (a) receive a single shopping cart and move the shopping cart through the processing station 22; (b) receive multiple shopping carts that are sufficiently separated from one another on the conveyor system so that no “un-nesting” is needed and post treatment nesting may not be possible, and simultaneously move the shopping carts relative to the processing station 22; (c) receive a string of nested shopping carts, “un-nest” the shopping carts so that surfaces of each of the shopping carts that would not otherwise be sufficiently exposed for receiving the disinfecting treatment are exposed in time to receive the treatment, nest the treated shopping carts, and simultaneously move the shopping carts relative to the processing station 22. With respect to any of these ways of using the conveyor system 38, the staging bar assembly 76 may or may not be utilized. However, the staging bar assembly 76 is most likely to be used when the conveyor system 38 is used to process a sting of nested shopping carts when the string of nested shopping carts is built up over time.

It should be appreciated that other conveyor systems that are capable of “un-nesting” and nesting operations and that utilize belts that move at different speeds are feasible. For instance, a conveyor system that utilizes a high-speed conveyor belt located between two, low speed conveyor belts is feasible. The belts can overlap with one another and have elevational differences that facilitate the transfer of a shopping cart from one belt to another, as in the conveyor system 38. Alternatively, the belts can be placed end-to-end. In this case, elevational differences between the belts are unnecessary. Further, if necessary to prevent a shopping cart from getting stuck in the “valley” between the ends of the end-to-end conveyor belts, a rod or bar can be positioned in the “valley.” Another approach to address the “valley” between conveyors positioned end-to-end is for the second conveyor belt (i.e., the belt to which a cart is being transition) to have a “bumpy” surface that can engage the crossbar of a shopping cart that is positioned in the “valley” and thereby facilitate the transition of the shopping cart from the first conveyor belt to the second conveyor belt. Yet another approach for addressing the “valley” between end-to-end conveyors is have the first conveyor positioned so that it slopes downward towards the second conveyor. Yet a further alternative to the placement of the belts overlaps two of the three belts and situates two of the three belts end-to-end. Also feasible is a conveyor system that utilizes a first pair of conveyors (one high speed and the other low speed) to facilitate “un-nesting” and a second pair of conveyors (one high speed and the other low speed) to facilitate nesting. Again, overlapping, end-to-end and combinations of overlapping and end-to-end belts are feasible.

It should also be appreciated that although the conveyor system 36 is capable of both “un-nesting” and nesting operations, certain applications may only require a conveyor system that is capable of facilitating one of the “unnesting” and nesting operations. For example, a conveyor system that is only capable of “un-nesting” untreated shopping carts would be appropriate when the user does not want the treated carts to be nested or the treated carts are nested in some other manner (e.g., by gravity). Similarly, a conveyor system that is only capable of “nesting” treated shopping carts would be appropriate when the carts are fed into the conveyor system such that the untreated carts are separated from one another by a distance that makes further separation unnecessary. In either case, a conveyor system that employs two belts that move at different speeds can be employed to achieve the desired nesting related function, i.e., “un-nesting” or nesting. The belts can be disposed end-to-end or overlap. If the belts overlap, the end points of the conveyor belt systems can be adjusted relative to those shown in FIG. 3A so that the conveyor belt systems are no longer than needed to accomplish the “un-nesting” or nesting operation.

It should be further appreciated that a conveyor system that uses belts that move at different speeds to perform nesting and/or un-nesting operations is not limited to any particular treatment station but can be used with many different types of treatment stations.

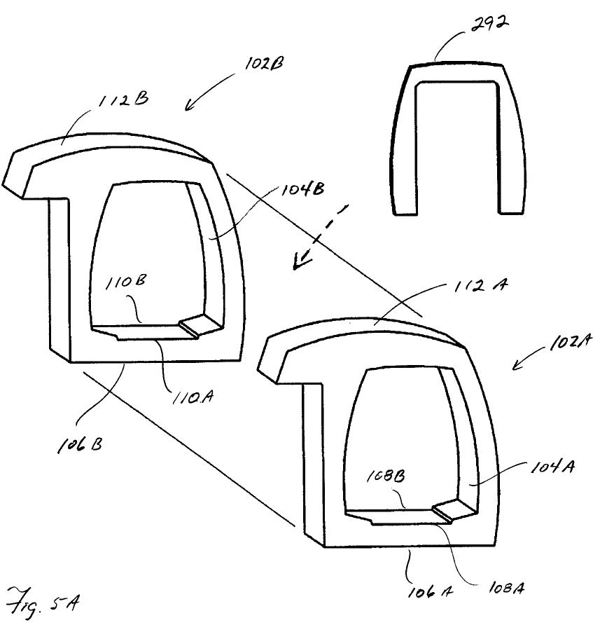

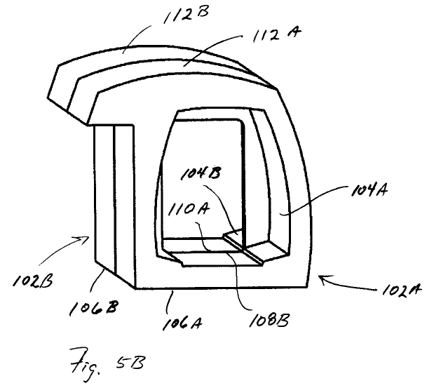

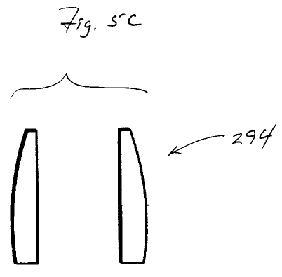

With reference to FIGS. 1A-1B, the treatment station 22 is comprised of three modular units 100A-100C. The modular units 100A-100B are for use in applying a liquid disinfectant to a shopping cart. The modular unit 100C is for use in blowing warm air onto a shopping cart to which a liquid disinfectant has been applied to dry the shopping cart. The modularity of the units 100A-100C is realized by designing each of the units 100A-100C such that each unit defines a portion of the pathway 30 and that the portion of the pathway that each unit defines can be readily aligned with a portion of the pathway defined by another unit. The ability to readily align the portions of the pathway defined by each of the units is achieved at least in part by, when two units are positioned on a reference surface, defining an end of the portion of the pathway defined by one unit and an end of the portion of the pathway defined by the other unit so that the ends are capable of being juxtaposed so as to form a greater portion of the pathway. In the illustrated embodiment, the ability to readily align the portions of the pathway is achieved by defining an end of a portion of the pathway defined by one unit and an end of the portion of the pathway defined by the other unit so that when the units are situated on a flat surface, the ends are capable of being brought together to form a greater portion of the pathway. This is illustrated with respect to FIGS. 5A-5B, which shows first and second modular units 102A, 102B. The first and second modular units 102A, 102B respectively comprise first and second pathway defining surfaces 104A, 104B and first and second flat, ground contact surfaces 106A, 106B. The first pathway defining surface 104A comprises first and second ends 108A, 108B and the second pathway defining surface 104B comprises first and second ends 110A, 110B. As shown in FIG. 5B, when the first and second modular units 102A, 102B are brought together such that the first and ground contact surfaces 106A, 106B are co-planar, the second end 108B of the first modular unit 102A and the first end 110A of the second modular unit 102B are capable of being aligned so as to form a greater portion of the pathway than was defined by either unit separately. Typically, the modular units 102A, 102B will not be positioned on a perfectly flat surface. To address this possibility, each of the units comprises adjustable legs (not shown) that are associated with the ground contact surfaces 106A, 106B. The first and second modular units 102A, 102B also respectively comprises first and second outer surfaces 112A, 112B that are substantially identical to one another. As a consequence, when the first and second units 102A, 102B are brought together, as shown in FIG. 5B, the first and second outer surfaces 112A, 112B form a larger outer surface that has the same shape as the first and second outer surfaces 112A, 112B.
it should be appreciated that, while the treatment station 22 shown in FIGS. 1A-1D is comprised of the three modular units 100A-100C, a treatment station comprised of two or more modular units is feasible. Additionally, modularity allows units that perform different functions to be designed and readily concatenated with other units to form a treatment station that meets the requirements for a particular application. Further, if desired, modules with “keyed” end surfaces that only allow modular units to be joined to one another in a specific sequence are feasible.

Further, while the first and second pathway defining surfaces 104A, 104B are shown as being closed-loop surfaces, it should be appreciated that modules with pathway defining surfaces that are not closed-loop surfaces are feasible. Stated differently, modules are feasible that when joined to one another do not form a tunnel-like pathway along which the shopping carts pass but form a pathway that is exposed to the environment. It should also be appreciated that a treatment station comprised of modular units is capable of being used with or adapted for use with a transport system other than a transport system that employs conveyors that move at different speeds.

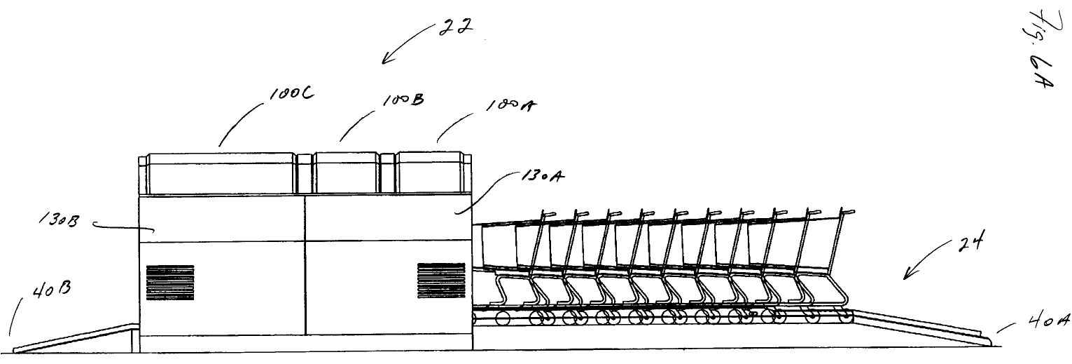

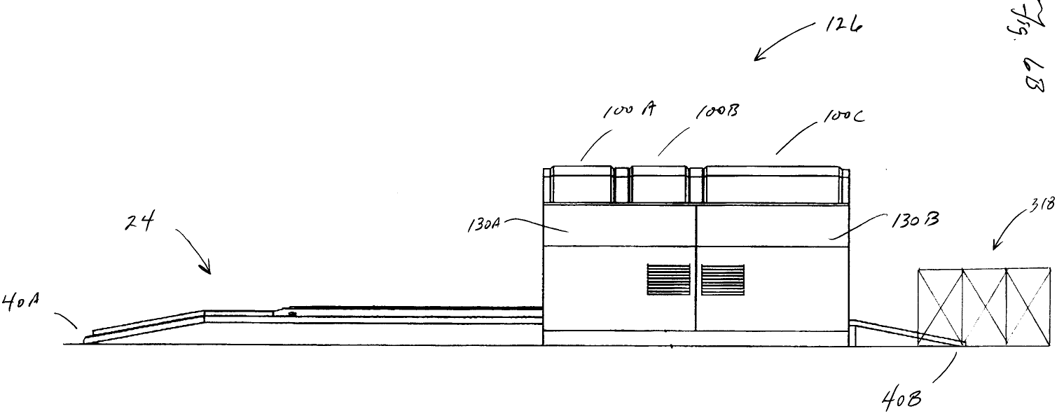

The interface structures of at least two of the two or more modular units comprising a treatment station are symmetrical with respect to a lateral plane that is located midway between the ends of the modular units and that is substantially perpendicular to the direction in which shopping carts move along the pathway. As a consequence, the modular units have interface symmetry that allows the unit to be concatenated with one another in a number of different sequences. With reference to FIG. 1B, the symmetry of the modular unit 100C is described. Modular unit 100C is comprised of first and second ends 116A, 116B and an interior surface that defines a portion of the pathway 30 (which are comparable to the first and second ends 108A, 108B and the first pathway defining surface 104A of the first modular unit 102A). The portion of the pathway 30 defined by the interior surface comprises a floor surface 118. A lateral plane 120 is located midway between the first and second ends 116A, 116B and substantially perpendicular to a direction 122 in which shopping carts move along the pathway 30. The end 116A and the end 116B are each capable of being readily aligned with other modular units and are symmetrical relative to the lateral plane 120. Consequently, the modular unit 100C is capable of taking different positions relative to other modular units in a treatment station. The other modular units 100A, 100B also have this symmetrical interface structure characteristic. As a consequence, these units are also capable of taking different positions relative to other modular units in a treatment station. For instance, FIG. 6A shows the treatment station 22 with a right-to-left sequence of modular units of modular unit 100A, modular unit 100B, and modular unit 100C. With this right-to-left sequence of modular units, the treatment station 22 is set up to receive untreated shopping carts from the right-hand side. FIG. 6B, in contrast, shows a treatment station 126 with a right-to-left sequence of modular unit of modular unit 100C, modular unit 100B, and modular unit 100A. With this right-to-left sequence of modular units, the treatment station 126 is set up to receive untreated shopping carts from the left-hand side. As can be appreciated, the ability to create a treatment station that facilitates either right or left hand entry provides flexibility in locating a treatment system that employs modules with symmetrical interface structures. Again, it should be appreciated that, while the treatment station 22 shown in FIGS. 1A-1D is comprised of the three modular units 100A-100C that each have the symmetrical interface characteristic, a treatment station comprised of two or more modular units is feasible.

One or more internal elements (i.e., a structure located between the ends of the module) of a module comprising a treatment station are also symmetric relative to a midlateral plane. Among these internal elements are those elements that if not symmetric relative to the plane would affect a shopping cart differently during operation of the system depending upon whether the cart entered the module unit through one end or the end of the module. In the case of the modular unit 100C, which is used in drying carts that have been treated with a liquid disinfectant, the elements of the unit that are symmetric relative to the plane 120 are those elements that if not symmetric relative to the plane would affect a shopping cart differently during operation of the system depending upon whether the shopping cart entered the unit through the first end 116A or the second end 116B. One element of the modular unit 100C that is symmetrically located is the group of nozzles that are used to disperse air onto treated shopping carts to dry the carts. If the group of nozzles was not symmetrically located relative to the plane, shopping carts would be engaged by the air stream projected by the nozzles at different times depending upon whether the cart entered the modular unit 1003 from the first end 116A or the second end 116B.

At least one of the modular units associated with a treatment station comprises a one-piece, molded plastic structure that comprises two or more elements needed to implement that function of the modular unit in a treatment station. For instance, a modular unit for use in applying a liquid disinfectant to a shopping cart may comprise a one-piece, molded plastic structure that comprises: (a) an enclosure that shields a shopping cart from wind, rain and other environmental factors that could adversely affect the application of a disinfectant to the cart; (b) a reservoir for holding the disinfectant that is to be applied to a cart; and (c) a structure for use in dispersing disinfectant onto a cart, such as a hanger that supports a manifold that is used to disperse disinfectant onto a cart. A modular unit for use in applying warm air to a shopping cart to dry the shopping cart after the application of a liquid disinfectant to the shopping cart may comprise a one-piece, molded plastic structure that comprises: (a) an enclosure that shields a shopping cart from wind, rain and other environmental factors that could adversely affect the application of air to the cart; and (b) one or more nozzles that are used to apply air to a cart. The use of a one-piece, molded plastic structure to realize two or more element reduces the part count of a module and, in some cases, renders the module less susceptible to vandalism.

Potentially associated with a modular unit is a component cabinet for housing certain components that are used in conjunction with the modular unit to provide a function associated with treating a shopping cart. With reference to FIGS. 1A-1D, a first cabinet 130A is associated with the modular units 100A-100B and a second cabinet 130B is associated with modular unit 100C. Since the modular units 100A, 100B serve to facilitate the application of a liquid disinfectant to shopping carts, the first cabinet 130A typically houses components that are used in the application of the liquid disinfectant. For instance, the first cabinet 130A may house a pump that is used to move disinfectant from a reservoir to a dispersal system, a control system for controlling the operation of the pump and possibly controlling other elements of the system 20 (e.g., the transport system 24), and an operator interface that allows an operator to interact with the control system. The modular unit 100C serves to facilitate the application of high-pressure air to shopping carts that have been treated with a liquid disinfectant to dry the carts. Consequently, the second cabinet 216B houses components that are used in the application of high-pressure air to the carts. For instance, the second cabinet 216B may house a heater-blower device, a control system for controlling the operation of the heater-blower device and possibly other elements of the system, and an operator interface that allows an operator to interact with the control system. It should be appreciated that a component cabinet can be associated with only one modular unit or with multiple modular units. Moreover, a component cabinet associated with one modular unit does not necessarily need to be attached to that modular unit. Further, if a cabinet is associated with a modular unit and attached to the modular unit, the cabinet is designed so as not to interfere with the modular features of the modular unit. Similarly, if a cabinet is associated with two or more modular units that are joined with one another or are to be joined with one another and the cabinet is to be attached to the joined modular units, the cabinet is designed so as not to interfere with any modular features of the joined modular units.

One or more modular units can be combined with components that are not integral to a modular unit and one or more enclosures to form a treatment system for use in a treatment station. A treatment station is comprised of one or more treatment systems. If a treatment station is comprised of two treatment systems, the systems can be modular and have symmetrical interfaces that allow the systems to be concatenated in different orders. For example, the treatment system 22 is comprised of: (a) a liquid disinfectant delivery system comprised of modular units 100A-100B, enclosure 130A, and related components; and (b) a drying station comprised of modular unit 100C, enclosure 130B, and related components. As shown in FIGS. 6A-6B, these two systems each have interface symmetry that allows them to be concatenated in two different orders. It should be appreciated that a modular unit that lacks an enclosure and/or lack components other than the components provided by the modular unit may, in certain applications, constitute a treatment system.

The following describes a number of embodiments of systems that are suitable for use in a treatment station. Each system is comprised of at least one modular unit, an enclosure, and related components that are not integral to a modular unit. Each modular unit has interface symmetry. As a consequence, each system has interface symmetry. Further, each modular unit is comprised of a molded plastic structure that provides at least two components or elements relating to the function of the module.

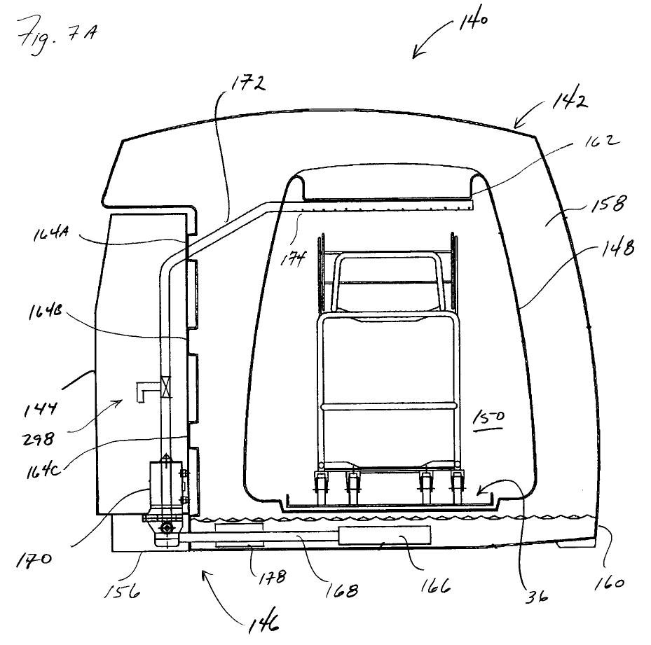

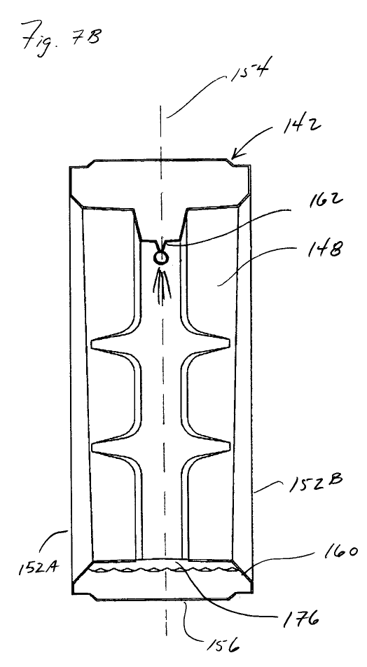

With reference to FIGS. 7A-7B, an embodiment of a liquid disinfectant delivery system 140 is described. The liquid disinfectant delivery system 140 is comprised of: (a) a modular unit 142 that is designed to facilitate the application of a liquid disinfectant to a shopping cart; (b) an cabinet 144 for housing components associated with applying a liquid disinfectant to a shopping cart; and (c) a closed-loop, liquid disinfectant transport system 146 for moving liquid disinfectant from a reservoir to a structure for dispersing disinfectant onto a shopping cart.

The modular unit 142 comprises a pathway defining surface 148 that defines a pathway 150. The pathway of 150 of modular unit 142 is capable of being readily aligned with the pathway associated another modular unit (as exemplified in FIGS. 5A-5B) to form a greater portion of a pathway of a treatment station comprised of modular units, such as treatment station 22. In this regard, the pathway defining surface 148 comprises first and second ends 152A, 152B that are symmetrical about a lateral plane 154 that is located midway between the first and second ends 152A, 152B. The modular unit 142 further comprises a substantially flat, ground contact surface 156. When the substantially flat, ground contact surface 156 is co-planar with the substantially flat ground contact surface of another modular unit, at least one of the first and second ends 152A, 152B can be aligned with an end that is associated with the other modular unit.

The modular unit 142 comprises: (a) an enclosure 158 for shielding a shopping cart from environmental factors that could adversely affect the application of disinfectant to a shopping cart; (b) a reservoir 160 for holding the liquid disinfectant that is to be applied to a cart; and (c) a hanger 162 for supporting a perforated tube that is used to disperse the liquid disinfectant onto a shopping cart. The enclosure 158, reservoir 160, and hanger 162 are each symmetrical about the lateral plane 154. Further, the modular unit 142 is a one-piece plastic structure that provides the enclosure 158, reservoir 160 and hanger 162.

The cabinet 144 houses components of the closed-loop, liquid disinfectant transport system 146 and is attached to the modular unit 142 at mounting points 164A-164C.

The closed-loop, liquid disinfectant transport system 146 for moving liquid disinfectant from a reservoir to a structure for dispersing disinfectant onto a shopping cart is comprised of: (a) a filter 166 located in the space defined by the reservoir 160; (b) a suction tube 168 with a first end that is operatively attached to the filter 166 and a second end for operatively engaging a pump; (c) a low-pressure centrifugal pump 170 with an inlet for operatively engaging the second end of the suction tube 168 and an outlet for operatively engaging a spray tube; and (d) a spray tube 172 for dispersing liquid disinfectant onto a shopping cart and having one end operatively connected to the outlet of the pump 170 and a second end that is operatively connected to the hanger 162. The pump 170 is substantially located within the cabinet 144. Further, the inlet of the pump 170 is located adjacent to the ground contact surface 156 to assure that, during operation, the inlet is below the surface of the liquid disinfectant held in the reservoir 160. In operation, the pump 170 pulls liquid disinfectant out of the reservoir 160 via the filter 166 and suction tube 168 and pushes the liquid disinfectant drawn out of the reservoir 160 through the spray tube 172. The liquid disinfectant is discharged through rations 174 in the spray tube 172 and onto a shopping cart located between the first and second ends 152A, 152B. Liquid disinfectant that either does not engage a shopping cart or drips off of a shopping cart passes through holes in the floor structure and a hole 176 defined by the reservoir 160 to be returned to the reservoir 160 and reused. Consequently, the system 140 operates to recycle the disinfectant.

It should also be appreciated that the centrifugal pump 170 and the location of the inlet of the centrifugal pump 170 below the surface of the disinfectant facilitates self-draining of the system 140 when the system is not in operation. To elaborate, when the pump 170 is deactivated, gravity forces the disinfectant remaining in the spray tube 172 to drain back through the pump 170 and into the reservoir 160. Some of the disinfectant remains in the pump 170. As a consequence, the pump 170 is “primed” for subsequent operation. The self-draining feature is particularly beneficial in cold weather applications because the disinfectant is isolated to the reservoir 160 and the pump 170, which has fairly intimate thermal contact with the disinfectant in the reservoir 160 via the suction tube 168. Consequently, to prevent damage to the system 140, it is only necessary to prevent freezing of the disinfectant present in the reservoir 160 and the pump 170. If this were not the case, freezing would need to be prevented throughout the system. In some applications, the thermal mass of the liquid disinfectant is sufficient to keep the disinfectant in the reservoir 160 and the pump 170 from freezing. However, in other applications, it is necessary to heat the disinfectant. For such applications, the system 140 further comprises a heating element 178 for heating the disinfectant in the reservoir. If needed, a heat sink can be attached to the suction tube 168 to conduct heat from the reservoir to the pump 170. The heating element 178 is also useful in applications in which freezing of the disinfectant is of little or no concern. In such applications, the heating element 178, by keeping the disinfectant at or above a certain temperature, promotes the sanitizing action of the disinfectant applied to the cartsand reduces the time needed to dry a cart.

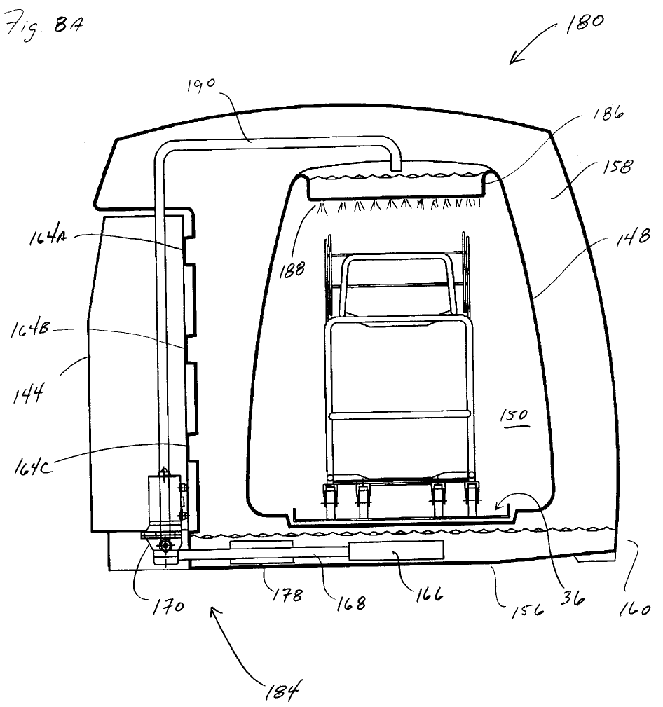

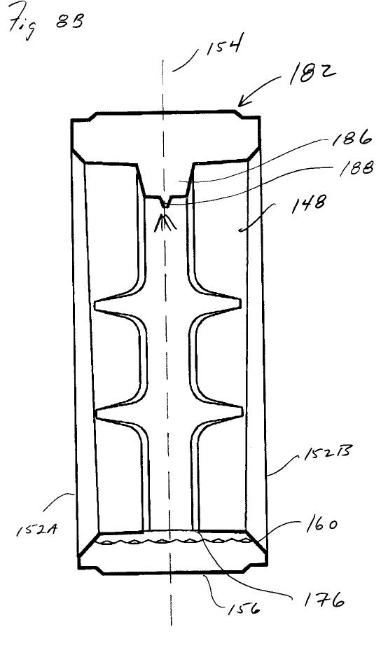

With reference to FIGS. 8A-8B, another embodiment of a liquid disinfectant delivery system 180 is described. With the exception of the structure used in transporting disinfectant from a low-pressure centrifugal pump to a dispersal mechanism and the dispersal mechanism, the system 180 is substantially identical to the system 140. As a consequence, elements of system 180 that are common to system 140 will bear the same reference numbers as the elements in system 140 and not be described further. The system 180 is comprised of: (a) a modular unit 182 that is designed to facilitate the application of a liquid disinfectant to a shopping cart; (b) a cabinet 144 for housing components associated with applying a liquid disinfectant to a shopping cart; and (c) a closed-loop, liquid disinfectant transport system 184 for moving liquid disinfectant from a reservoir to a structure for dispersing disinfectant onto a shopping cart.

The modular unit 182 comprises a reservoir 186 for receiving and storing disinfectant and a plurality of holes 188 for dispersing disinfectant from the reservoir 186 onto a shopping cart. The reservoir 186 and the holes 188 are symmetrical relative to the lateral plane 154. Otherwise, the modular unit 182 is substantially identical to the modular unit 142 associated with the system 140.

The cabinet 144 associated with the system 180 is substantially identical to the cabinet 144 associated with the system 140.

The closed loop, liquid disinfectant transport system 184 comprises a delivery tube 190 for transporting liquid disinfect from the pump 170 to the reservoir 186. Otherwise, the closed-loop transport system 184 is substantially identical to the system 146 associated with the system 140. In operation, the pump 170 pulls liquid disinfectant out of the reservoir 160 via the filter 166 and suction tube 168 and pushes the liquid disinfectant drawn out of the reservoir 160 through the delivery tube 190 to the reservoir 186. Liquid disinfectant stored in the reservoir 186 is discharged through holes 188 and onto a shopping cart located between the first and second ends 152A, 152B. Liquid disinfectant that either does not engage a shopping cart or drips off of a shopping cart passes through holes in the floor structure 36 and a hole 176 defined by the reservoir 160 to be returned to the reservoir 160 and reused. Consequently, the system 180 operates to recycle the disinfectant. The system 180 also provides self-draining and can include a heating element 178.

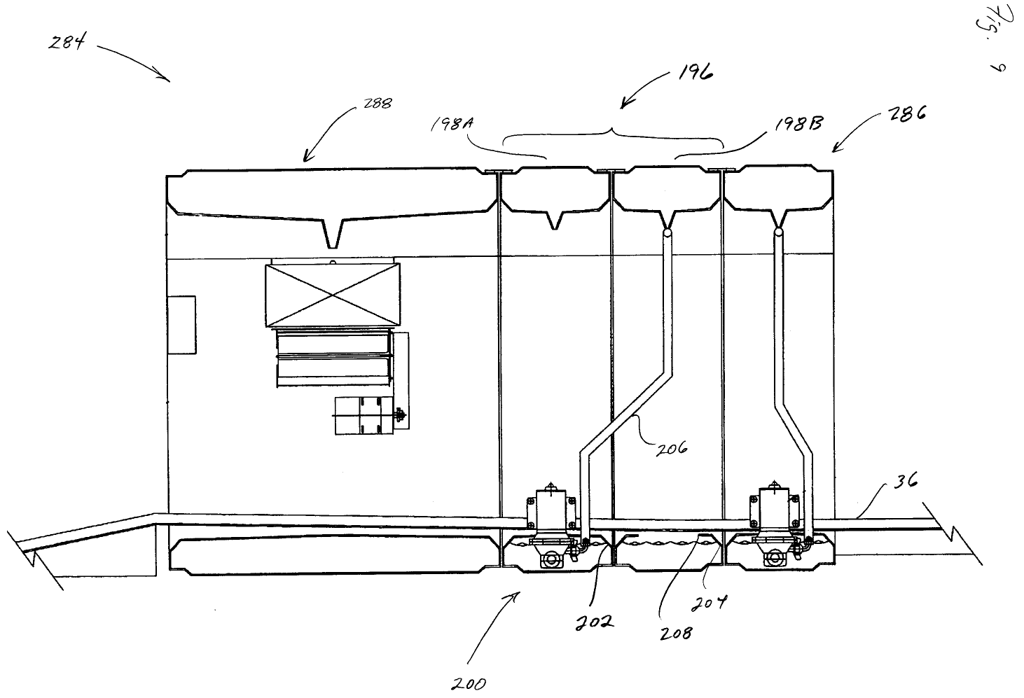

With reference to FIG. 9, an embodiment of a non-recycling liquid disinfectant delivery system 196 is described. The liquid disinfectant delivery system 196 is comprised of: (a) first and second modular units 198A, 198B that are designed to facilitate the application of a liquid disinfectant to a shopping cart; (b) a cabinet (not shown) for housing components associated with applying a liquid disinfectant to a shopping cart; and (c) a closed-loop, liquid disinfectant transport system 200 for moving liquid disinfectant from a reservoir to a structure for dispersing disinfectant onto a shopping cart.

The first and second modular units 198A, 198B are each substantially identical to the modular unit 142. A reservoir 202 associated with the first modular unit 198A is used to hold unused liquid disinfectant. A reservoir 204 associated with the second modular unit 198B is used to hold liquid disinfectant after the liquid disinfectant has been dispersed onto a shopping cart. The system 200 is used to pull unused liquid disinfectant from the reservoir 202 and push the disinfectant through a spray tube 206 that disperses the liquid disinfectant onto carts that are passing over the reservoir 204. Liquid disinfectant that either does not engage a shopping cart or drips off of the shopping cart passes through holes in the floor structure 36 and a hole 208 defined by the reservoir 204 to be collected in the reservoir 204. In other respects, the system 200 is substantially identical to the system 140 and will not be described further.

It should be appreciated that modular unit 182 could be used in place of either or both of first and second modular units 198A, 198B. Further, if modular unit 182 is used in place of modular unit 198B, the system 200 is modified so as be substantially identical to the liquid disinfectant transport system 184.

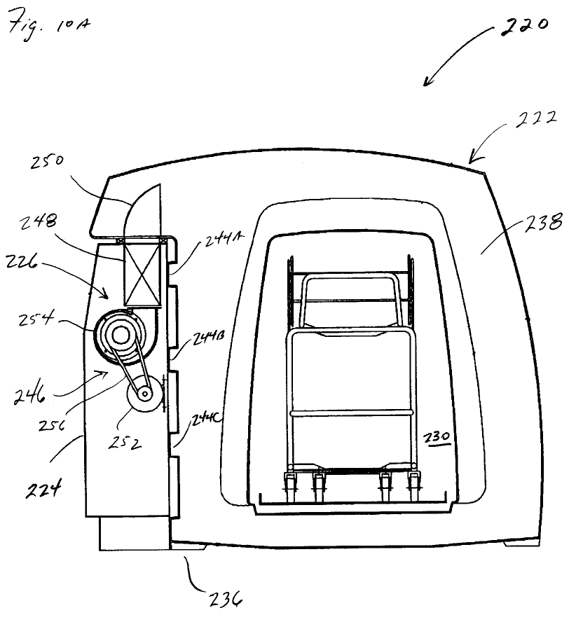

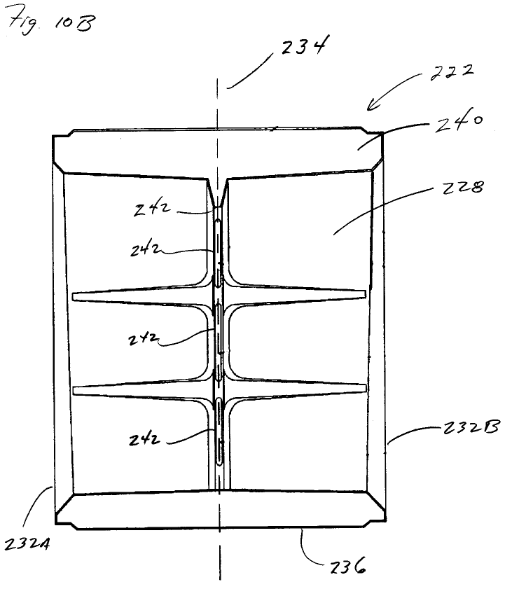

With reference to FIGS. 10A-10B, an embodiment of a drying system 220 is described. The drying system 220 is comprised of: (a) a modular unit 222 that is designed to facilitate the application of warm air to a shopping cart that has been treated with a liquid disinfectant to dry the shopping cart; (b) a cabinet 224 for housing components associated with applying warm air to a shopping cart; and (c) heater-blower device 226 for producing moving warm air.

The modular unit 222 comprises a pathway defining surface 228 that defines a pathway 230. The pathway of 230 of modular unit 222 is capable of being readily aligned with the pathway associated another modular unit (as exemplified in FIGS. 5A-5B) to form a greater portion of a pathway of a treatment station comprised of modular units, such as treatment station 22. In this regard, the pathway defining surface 228 comprises first and second ends 232A, 232B that are symmetrical about a lateral plane 234 that is located midway between the first and second ends 232A, 232B. The modular unit 222 further comprises a substantially flat, ground contact surface 236. When the substantially flat, ground contact surface 236 is co-planar with the substantially flat ground contact surface of another modular unit, at least one of the first and second ends 232A, 232B can be aligned with an end that is associated with the other modular unit.

The modular unit 222 comprises: (a) an enclosure 238 for shielding a shopping cart from environmental factors that could adversely affect the application of warm air to a shopping cart; (b) a plenum 240 for receiving warm air from the heater-blower device 226; and (c) a plurality of outlet nozzles 242 that receive warm air from the plenum 242 and direct the warm air onto a shopping cart. The plenum 242 assures that each of the nozzles 242 receives warm air at substantially the same pressure. The enclosure 238, plenum 242, and plurality of outlet nozzles 242 are each symmetrical about the lateral plane 234. Further, the modular unit 222 is a one-piece plastic structure that provides the enclosure 238, plenum 242, and plurality of nozzles 242.

The cabinet 224 houses components of the heater-blower device 226 and is attached to the modular unit 222 at mounting points 244A-244C.

The heater-blower device 226 is comprised of a blower 246, a heater 248, and a duct 250 for directing heated and blown air into the plenum 240. The blower 246 is comprised of an electric motor 252, a fan 254, and a fan belt 256 that couples the motor 252 to the fan 254. In operation, the blower 246 drives air across the heater 248, which heats the air, and through the duct 250 into the plenum 240. The plenum 240 then provides the pressurized and warm air to the plurality of nozzles 242, which direct the air onto any shopping cart that is in the passageway 230.

It should be appreciated that other kinds of treatment systems are feasible. For instance a treatment system that irradiates a shopping cart with electromagnetic radiation, such as ultraviolet light, is feasible.

As should be appreciated, a treatment system can be combined with other treatment systems to form a treatment station, such as treatment station 22. If a treatment station is comprised of two treatment systems, the systems can be modular and have symmetrical interfaces that allow the systems to be concatenated in different orders. For example, the treatment system 22 is comprised of: (a) a liquid disinfectant delivery system comprised of modular units 100A-100B, enclosure 130A, and related components; and (b) a drying station comprised of modular unit 100C, enclosure 130B and related components. As shown in FIGS. 6A-6B, these two systems each have interface symmetry that allows them to be concatenated in two different orders. It should be appreciated that a modular unit that lacks an enclosure and/or lack components other than the components provided by the modular unit may, in certain applications, constitute a treatment system.

The following describes a number of embodiments of a treatment station comprised of two or more treatment systems. Each system is comprised of at least one modular unit, an enclosure, and related components that are not integral to a modular unit. Each modular unit has interface symmetry. As a consequence, each treatment system has interface symmetry.

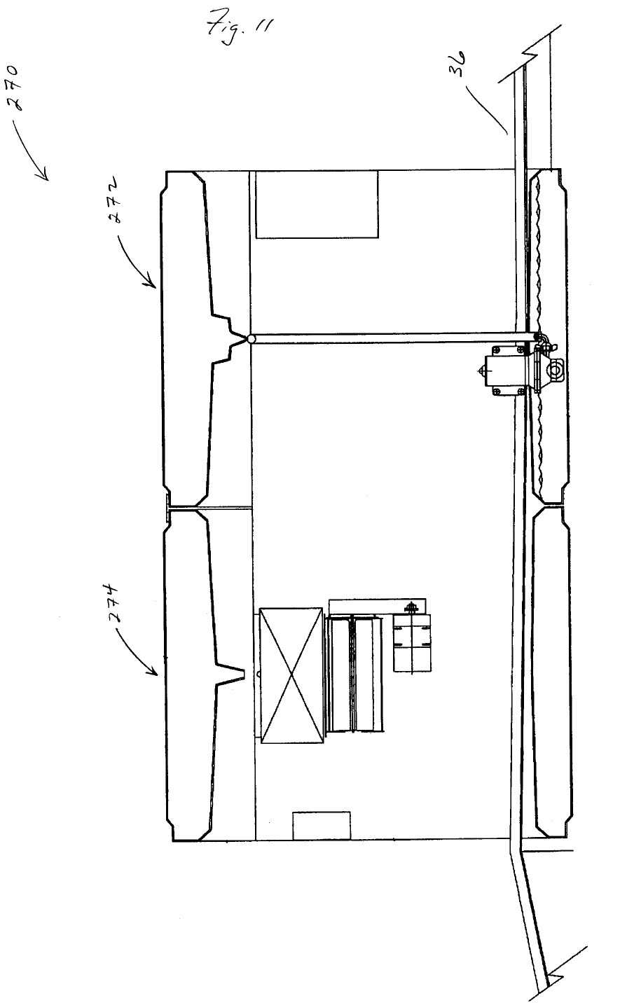

With reference to FIG. 11, an embodiment of a treatment station 270 is described. The treatment station 270 is comprised of: (a) a liquid disinfectant delivery system 272 that is substantially identical to one of liquid disinfectant delivery systems 140, 180; and (b) a dryer system 274 that is substantially identical to the dryer system 220.

With reference to FIG. 1A, treatment station 22 is comprised of (a) a liquid disinfectant system 278 that is comprised of modular units 100A, 100B; and (b) a dryer system 274 that is substantially identical to dryer system 220. The two modular units 100A, 100B and related components can implement a non-recycling disinfectant delivery system that is substantially identical to non-recycling disinfectant delivery system 196. Alternatively, each of the modular units 100A, 100B can be substantially identical to one of liquid disinfectant delivery system 140, 180. Such an implementation would allow a shopping cart to be treated with two different disinfectants.

With reference to FIG. 9, a treatment station 284 is described. Treatment station is comprised of: (a) a recycling liquid disinfectant delivery system 286 that is substantially identical to one of liquid disinfectant system 140, 180; (b) the non-recycling liquid disinfectant delivery system 196; and (c) a dryer system 288 that is substantially identical to the dryer system 220.

As can be appreciated from the foregoing, treatment stations that have two or more recycling liquid disinfectant delivery systems and/or two or more non-recycling liquid disinfectant delivery systems are feasible. Further, treatment stations with two or more dryer systems are feasible. Typically, at least one of the dryer systems would be located between two liquid disinfectant delivery systems. If open, air-drying of treated shopping carts is feasible, a treatment station without a dryer system is also feasible.

With reference to FIG. 5B, if the modular unit 112A is for use in a liquid disinfectant delivery system that disperses a liquid disinfectant onto a cart and modular unit 112B is for use in a dryer system that dries a shopping cart after a liquid disinfectant has been applied to the cart via modular unit 112A, the operation of the dryer system may blow air through the modular unit 112A that drives liquid disinfectant out of the modular unit 112A before the liquid disinfectant can disinfect a cart. To prevent this from occurring, an embodiment of treatment station comprises a baffle 292 that is interposed between the modular unit 112A and the modular unit 112B. Alternatively, with reference to FIG. 5C, a two-piece baffle 294 is employed.

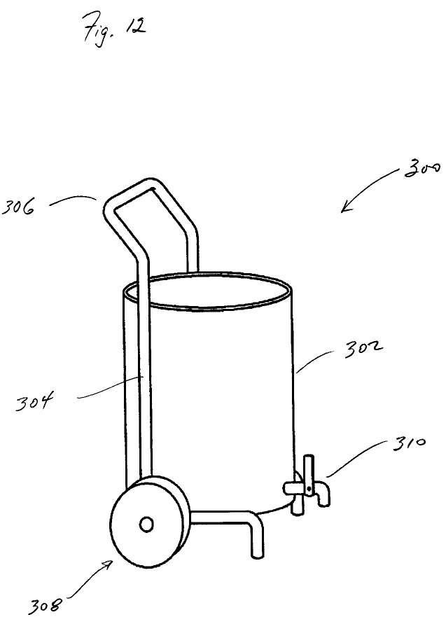

The removal of used liquid disinfectant from a liquid delivery system and replenishment of the system with new liquid disinfectant is occasionally necessary. To facilitate removal of used liquid disinfectant from a recycling liquid disinfectant delivery system, a diverter valve is placed on the outlet side of the pump so that the liquid disinfectant can be diverted away from the dispersal device and into a suitable container. With reference to FIG. 7A, the embodiment of a liquid disinfectant delivery system 140 comprises a diverter valve 298 that allows liquid disinfectant to be diverted from the spray tube 172 to a suitable container. With respect to the non-recycling liquid disinfectant delivery system 196 illustrated in FIG. 9, removal of used liquid disinfectant is accomplished using: (a) a first diverter valve, like diverter valve 298, that is located on the outlet side of the pump; and (b) a second diverter valve that is located on the inlet side of the pump and allows the pump to be switched between pumping unused liquid disinfectant from reservoir 202 or used liquid disinfectant from reservoir 204 via appropriate tubing. By appropriately setting the first and second diverter valves, the used liquid disinfectant can be removed from reservoir 204. With reference to FIG. 12, an embodiment of a container system 300 for transporting liquid disinfectant is described. The container system 300 is a comprised of a container body 302 for holding liquid disinfectant, a frame 304 that is attached to the container body 302, a handle 306 that is attached to or part of the frame 304, a pair of wheels 308 that are attached to the frame 304, and a spigot 310 that allows the container body 302 to be drained. The container system 300 is sized to fit within the cabinet 144. When spent disinfectant is to be removed from the system, the diverter valve 298 is actuated so that the spent disinfectant being pumped by the pump 170 flows into the container body 302. The container system 300 is used to transport the spent liquid disinfectant to a suitable disposal site where the spigot 310 is opened and the container body 302 is emptied of the spent disinfectant. Typically, the container body 302 will then be cleaned, filled with new liquid disinfectant, and transported back to the system. Once at the system, the spigot is opened adjacent to the hole 176 and the new disinfectant is discharged into the reservoir 160 via the hole 176.

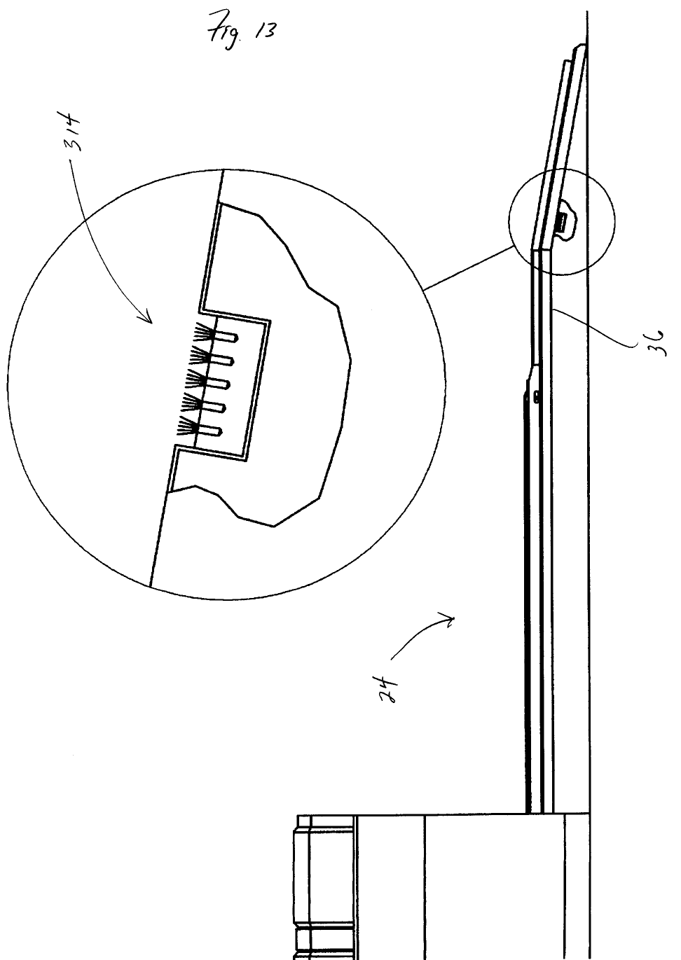

Often the wheels of shopping carts pick up particles, such as rock pebbles, from the parking lot and similar area. The particles frequently damage the floor of the store. With reference to FIG. 13, an embodiment of a system for disinfecting shopping carts also comprises a brush system 314 for removing particles from the wheels of shopping carts is described. The brush system 314 is comprised of one or more brush surfaces that move transverse to the direction in which the carts are moving over the floor structure 36. By having the brush surfaces move at an angle that is other than perpendicular to the direction in which the carts are moving, the brushes force the wheels to rotate so that most if not all of the floor contacting surface of each of the wheels is contacted by the brush.

With reference to FIG. 6B, if desired, a fence structure 318 can be located adjacent to the second floor end 40B to control and restrict the movement of treated shopping carts discharged from the treatment station 22.

In certain applications, the shopping carts that are to be processed by a system for disinfecting shopping carts may be particularly dirty. For instance, shopping carts may become caked with mud when used to transport goods to automobiles located in dirt parking lots that become muddy after a rain or snow storm. In such situations, it is desirable to remove the mud or other debris from the shopping cart and to do so before subjecting the cart to a disinfecting treatment. For such applications, the system for disinfecting shopping carts further comprises a power wash system before the first disinfectant system. On embodiment of the power wash system comprises a reservoir, a hand held spray wand for use by an operator, and pressurization system to pressurize the water for the spray wand. In another embodiment, a power spray station positioned adjacent to the transport system is used to power wash carts as they move along the transport system. For cold weather applications, the water can be treated with anti-freeze.

1. A system for disinfecting shopping carts comprising:
a treatment station for applying a disinfectant to a shopping cart;
wherein said treatment station defines an entry, an exit, and a pathway extending between said entry and exit and along which a shopping cart travels; and
a conveyor system for transporting shopping carts relative to said treatment station at a first speed and at a second speed that is different than said first speed so as to perform a shopping cart nesting related operation.

2. The system of claim 1, wherein said conveyor system comprises:
a first conveyor for moving a shopping cart at a first speed; and
a second conveyor for moving a shopping cart at a second speed that is greater than said first speed.

3. The system of claim 2, wherein:
said first conveyor extends from a first conveyor first end to a first conveyor second end; and
said second conveyor extends from a second conveyor first end to a second conveyor second end;
wherein during operation, shopping carts move from said first conveyor first end towards said first conveyor second end;
wherein during operation, shopping carts move from said second conveyor first end towards said second conveyor second end;
wherein said first and second conveyors are each located so as to define a travel path for a cart being transported by said first and second conveyors;
wherein when a cart travels along said travel path, said first conveyor first and second ends occur in the same order as said second conveyor first and second ends.

4. The system of claim 3, wherein:
said second conveyor first end is located between said first conveyor first and second ends.

5. The system of claim 4, wherein:
said first conveyor has a first surface for engaging a shopping cart;
wherein said first surface, at a location substantially adjacent to said second conveyor first end, has a first elevation; and
said second conveyor has a second surface for engaging a shopping cart;
wherein said second surface, at said location substantially adjacent to said second conveyor first end, has a second elevation that is different than said first elevation.

6. The system of claim 5, wherein:
said second elevation is greater than said first elevation.

7. The system of claim 5, wherein:
said location adjacent to said second conveyor first end is at a site along said travel path that when a shopping cart is traveling along said path, occurs before a disinfectant can be applied to the shopping cart.

8. The system of claim 4, wherein:
said first conveyor and said second conveyor are positioned so as to impart a rotation to a shopping cart during a transition of the shopping cart from said first conveyor to said second conveyor.

9. The system of claim 3, wherein:
said second conveyor second end is located between said first conveyor first and second ends.

10. The system of claim 9, wherein:
said first conveyor has a first surface for engaging a shopping cart;
wherein said first surface, at a location substantially adjacent to said second conveyor second end, has a first elevation; and
said second conveyor has a second surface for engaging a shopping cart;
wherein said second surface, at said location substantially adjacent to said second conveyor second end, has a second elevation that is different than said first elevation.

11. The system of claim 10, wherein:
wherein said second elevation is greater than said first elevation.

12. The system of claim 10, wherein:
said location adjacent to said second conveyor second end is at a site along said travel path that when a shopping cart is traveling along said path, occurs after a disinfectant can be applied to the shopping cart.

13. The system of claim 9, wherein:
said first conveyor and said second conveyor are positioned so as to impart a rotation to a shopping cart during a transition of the shopping cart from said second conveyor to said first conveyor.

14. The system of claim 3, wherein:
said second conveyor first and second ends are located between said first conveyor first and second ends.

15. The system of claim 14, wherein:
said first conveyor has a first surface for engaging a shopping cart;
wherein said first surface, at a first location substantially adjacent to said second conveyor first end, has a first elevation;
wherein said first surface, at said second location substantially adjacent to said second conveyor second end, has a second elevation;
said second conveyor has a second surface for engaging a shopping cart;
wherein said second surface, at said first location substantially adjacent to said second conveyor first end, has a third elevation;
wherein said second surface, at said second location substantially adjacent to said second conveyor second end, has a fourth elevation.

16. The system of claim 15, wherein:
said third elevation is greater than said first elevation; and
said fourth elevation is greater than said second elevation.

17. The system of claim 15, wherein:
said first location adjacent to said second conveyor first end is at a site along said travel path that when a shopping cart is traveling along said path, occurs before a disinfectant can be applied to the shopping cart; and
said second location adjacent to said second conveyor second end is at a site along said travel path that when a shopping cart is traveling along said path, occurs after a disinfectant can be applied to the shopping cart.

18. The system of claim 14, wherein:
said first conveyor and said second conveyor are positioned so as to impart a rotation to a shopping cart during a transition of the shopping cart between said first conveyor and said second conveyor.

19. The system of claim 2, wherein said conveyor system comprises:
a staging bar for moving a shopping cart between a first position that is separated from said first and second conveyor and a second position that allows one of said first and conveyors to engage a shopping cart.

20. A system for disinfecting shopping carts comprising:
a treatment station for applying a disinfectant to a shopping cart; and
a transport system for moving a shopping cart relative to said treatment station;
wherein said treatment station defines an entry, an exit, and a pathway extending between said entry and said exit and along which a shopping cart travels during operation of the system;
wherein said treatment station comprises a first modular unit and a second modular unit that is operatively attached to said first modular unit;
wherein said first modular unit defines a first portion of said pathway and comprises a first ground engagement surface;
wherein said second modular unit defines a second portion of said pathway and comprises a second ground engagement surface;
wherein when said first and second ground engagement surfaces are in contact with a substantially flat ground surface, said first and second portions of said pathway are substantially aligned.

21. The system of claim 20, wherein said first modular unit comprises:
a first side;
a second side that is separated from and substantially parallel to said first side;
wherein said first portion of said pathway, in the direction along which a shopping cart travels, extends substantially perpendicular to said first and second sides;
wherein a lateral plane is located midway between and substantially parallel to said first and second sides.

22. The system of claim 21, wherein:
said first modular unit comprises a hanger for supporting a conduit;
wherein said hanger is substantially symmetrical relative to said lateral plane.

23. The system of claim 21, wherein:
said first modular unit comprises a reservoir for holding a liquid disinfectant;
wherein said reservoir is substantially symmetrical relative to said lateral plane.

24. The system of claim 21, wherein:
said first modular unit comprises a baffle system for preventing liquid disinfectant from moving outside of said first modular unit.

25. The system of claim 21, wherein:
said first modular unit comprises an air nozzle structure for directing air onto a shopping cart after a liquid disinfectant has been applied to the shopping cart;
wherein said air nozzle system is substantially symmetrical relative to said lateral plane.

26. The system of claim 20, wherein:
said first modular unit is adapted to apply a liquid disinfectant to a shopping cart.

27. The system of claim 20, wherein:
said first modular unit is adapted to applying a first liquid disinfectant to a shopping cart; and
said second modular unit is adapted to apply a second liquid disinfectant to a shopping cart.

28. The system of claim 20, wherein:
said first modular unit is adapted to apply a liquid disinfectant to a shopping cart; and
said second modular unit is adapted to provide said liquid disinfectant to said first modular structure.

29. The system of claim 20, wherein:
said first modular unit is adapted to apply a liquid disinfectant to a shopping cart; and
said second modular structure is adapted to apply moving air to a shopping cart after a liquid disinfectant has been applied to the shopping cart.

30. A system for disinfecting shopping carts comprising:
a treatment station for applying a disinfectant to a shopping cart; and
a transport system for moving a shopping cart relative to said treatment station;
wherein said treatment station defines an entry, an exit, and a pathway extending between said entry and said exit and along which a shopping cart travels during operation of the system;
wherein said treatment station comprises a molded structure.

31. The system of claim 30, wherein:
said molded structure comprises an enclosure that defines at least a portion of said pathway and a liquid disinfectant application structure.

32. The system of claim 31, wherein:
said liquid disinfectant application structure comprises a hanger for supporting a conduit that is used to transport liquid disinfectant.

33. The system of claim 31, wherein:
said liquid disinfectant application structure comprises a reservoir for holding a liquid disinfectant.

34. The system of claim 31, wherein:
said liquid disinfectant application structure comprises a baffle system for preventing liquid disinfectant from moving beyond a defined space.

35. The system of claim 30, wherein:
said molded structure comprises an enclosure that defines at least a portion of said pathway and an air application structure.

36. The system of claim 35, wherein:
said air application structure comprises an inlet for mating with the outlet of a blower.

37. The system of claim 35, wherein:
said air application structure comprises an outlet nozzle for directing air onto a shopping cart after a liquid disinfectant has been applied to the shopping cart.

38. The system of claim 30, wherein:
said molded structure comprises an enclosure that defines at least a portion of said pathway.

39. The system of claim 30, wherein:
said molded structure comprises a hanger for supporting a conduit for conveying a liquid disinfectant.

40. The system of claim 30, wherein:
said molded structure comprises a baffle system for preventing liquid disinfectant from moving beyond a defined space.

41. The system of claim 30, wherein:
said molded structure comprises a reservoir for holding a liquid disinfectant.

42. The system of claim 30, wherein:
said molded structure comprises an air inlet for mating with the outlet of a blower.

43. The system of claim 30, wherein:
said molded structure comprises an outlet nozzle for directing air onto a shopping cart after a liquid disinfectant has been applied to the shopping cart.

44. A system for disinfecting shopping carts comprising:
a treatment station for applying a disinfectant to a cart; and
a transport system for moving a shopping cart relative to said treatment station;
wherein said treatment station defines an entry, an exit, and a pathway extending between said entry and said exit and along which a shopping cart travels during operation of the system;
wherein said treatment station comprises:
a reservoir for holding a liquid disinfectant;
an application structure for dispensing liquid disinfectant into at least a portion of said pathway; and
a low-pressure pump for moving liquid disinfectant from said reservoir to said application structure.

45. The system of claim 44, further comprising:
a passageway between said pathway and said reservoir that allows at least a portion of the liquid disinfectant that has been dispensed into said pathway to return to said reservoir.

46. The system of claim 44, wherein:
said low-pressure pump comprises a centrifugal pump.

47. The system of claim 46, wherein:
said centrifugal pump is located at substantially the same elevation above the ground as said reservoir when the system is in an operational configuration.

48. The system of claim 44, further comprising:
a heating element located within said reservoir.

49. The system of claim 44, further comprising:
a pump inlet conduit extending from said low-pressure pump into said reservoir; and a heat sink attached to said pump inlet conduit for transferring heat from said reservoir to said low-pressure pump.

50. The system of claim 44, wherein:
said application structure comprises a conduit.

51. The system of claim 44, wherein:
said application structure comprises a manifold for holding liquid disinfectant conveyed from said reservoir by said low-pressure pump.

52. The system of claim 44, further comprising:
a baffle system for preventing liquid disinfectant from moving beyond a defined space.

53. The system of claim 44, further comprising:
a removable receptacle for receiving liquid disinfectant from said reservoir that is to be disposed.

54. The system of claim 44, further comprising:
a wash station for washing a shopping cart before the shopping cart passes said entry of said treatment station.

55. The system of claim 54, wherein:
said wash station comprises a hand-held wand for use in spraying a shopping cart.

56. The system of claim 44, further comprising:
a brush device for removing particles from the wheels of a shopping cart.

57. The system of claim 44, wherein:
said brush device comprises a brush and an electrical actuator for producing a motive force for moving said brush.
